(19) United States
(12) Patent Application Publication
(10) Pub. No.: US 2004/0237967 A1
(43) Pub. Date: Dec. 2, 2004

(54) Nasal breathing apparatus

(76) Inventor: Ralph A. Davis, Wichita, KS (US)

Correspondence Address: Bradley P. Sylvester, Suite 300, 200 N. Broadway, Wichita, KS, 67202, US

(21) Appl. No.: 10/448,858

(22) Filed: May 31, 2003

(51) Int. Cl. A61M 16/00

(52) U.S. Cl. 128/207.18; 128/200.26; 128/200.24; 128/207.13; 128/206.27

(57) ABSTRACT

An improved breathing devise, for use in the nasal passageways. This nasal breathing apparatus utilizes a flexible tube, that defines two centrally adjacent notches, spaced apart by a central tube portion. The terminating ends of the tube preferably have a portion of the circumferential tube removed, so that the ends define curved flanges. The section of modified tubing is bent approximately 90 degrees from the central portion, with the arcual outer surfaces of the tube ends being placed directly into the nasal passageways. The flexible tube will urge the nasal passageway walls apart, thereby increasing the overall diameter of said passageways. Airflow is not significantly restricted, due to the tubular configuration of the apparatus, which conforms to the nasal passageway shape it contacts. The central tube portion is capable of receiving an aromatic plug, which releases chemical substances adjacent to the nasal openings. This removable plug allows the administration of time-release medication, or simply provides a pleasant aroma for the wearer. A strap may be used to keep the nasal breathing apparatus in position during sleeping.

This novel breathing apparatus is derived from alterations made to a single tubular structure, where a tube section is placed between two opposing hinge plates, which define the appropriate notches. The hinge plates also only grip a portion of the width of the tube at its ends. The notches and flanges in the tubing are created by removal of all exposed tubing that is not between the hinge plates.

### DESCRIPTION OF PRIOR ART

Referring now to U.S. Pat. No. 5,727,543 (Corsaro), a nasal breathing device is shown, in which a shaped wire is formed so as to be able to be inserted into the nasal passages. This configuration allows the wire structure to effectively brace the walls of the nasal passage away from the nasal septum. There is little spring action involved in the use of this apparatus, which appears to rely on the structure providing the bracing necessary to keep the nasal passages open.

Referring now to U.S. Pat. No. Des. 4,220,800, a design for an apparatus having a use similar to the Corsaro patent is shown. This is a rigid structure, and does not appear to rely on spring action to keep the nasal passages open.

Referring now to U.S. Pat. No. 6,238,411 (Thorner), and internal nasal dilator is shown. This intervention appears to be an attempt to maintaining the dilation of the nasal passages in a normal configuration, as opposed to enhancing the amount of air flow from a stable ordinary condition. This invention uses a combination of internal and external forces to affect the nasal passages, and uses a sheet of flat bendable spring material internally. Since a flat material is used, the apparatus itself must extend down well below the nasal openings, so as not to block the air flow. This extension can present a problem in the use of the device, since it is more easily blocked or dislodged.

Referring now to U.S. Pat. No. 6,270,512 (Rittman), an internally secured nasal dilator is shown, in which the improvements over the prior art appears to be that a three-dimensional framework is shown. The framework itself forcibly causes the nasal passage to maintain a specific circumferential size.

Other prior art has been directed to external methods at increasing or maintaining nasal passage opening size. U.S. Pat. No. 6,276,360 (Cronk et al.) defines an external nasal dilator which has been impregnated with aromatic substances, or has had them placed upon the physical structure, so as to also provide a means whereby aromatic odors are in proximity to the nasal openings. One chief drawback to this invention, with regard to the aromatic feature, is that the majority of the aromatic distribution is lost as a result of typical atmospheric dispersal. The aromatic benefits are not immediately adjacent to the nasal openings.

Another creative attempt tat maintaining nasal passage opening size is shown in U.S. Pat. No. 6,540,766 (Martino), in which an apparatus is directed for specific use with horses. This specific invention relies on the adjustment means or elastic means that pulls the apparatus toward the ears. It is unlikely that a similar apparatus would be voluntarily used with humans.

### BRIEF DESCRIPTION OF THE PREFERRED EMBODIMENT

This invention is intended to provide suitable manner in which the nasal passages of a person are increased in their internal circumference to a maximum size. This has the benefit of assisting persons with general breathing difficulty, as well as providing a preventive means to discourage snoring.

The nasal breathing apparatus comprises a tubular structure, where two centrally located notches are defined on one viewable side of the tubing, with the other side, being 180 degrees of circumference around the tubing, having a portion of the side walls removed, so that the end portions of the tube comprise arcual flanges. The type of tubing used is preferably a flexible but resilient material, that will return to its original configuration if allowed to do so.

The set of notches de fined on the tubing serve four purposes. First, they allow the end portions of the tubing to be flexed or bent along the length of the tubing at the point of the notches, with the end portions being flexed or bent approximately 90 degrees in relation to the central portion of tubing between the two notches. Without said notches, the tubing walls would deform appreciably and be difficult to flex or bend at the appropriate positions along the length of the tubing.

The length of the tubing between the two notches is referred to as the “central portion”. This serves as a bridge between the end flange portions that are placed into the person's nasal passages. The second benefit offered by the notches is that access is provided to the central tube portion when the flanges are bent, so that a restraining strap or cord may be placed through the central tube portion, with the entry and exit points of the strap or cord in and out of the tubing being through the respective notches.

A third purpose of the notches is that they provide an opening to the rest of the tubing that comprises each respective end portion, so that when the end portions are flexed or bent in relation to the central tubing, air flow through the end portions of the tubing is unrestricted, as the notches allow air to freely flow through the bent or flexed end portions.

The fourth benefit derived from the notches is that they allow access to the central tube portion so that in addition to any cord or strap, an aromatic capsule or plug may be inserted into the central tube portion. This allows any vapors or chemical odors to have an optimal position regarding the air that enters the nasal passages.

The end portions of this nasal breathing apparatus comprise arcual sided flanges. These flanges provide a comfortable shape and surface, which minimizes discomfort as they contact the nasal passageways opposite of the nasal septum. When the nasal breathing apparatus is placed within each of the person's nasal passageways, the flanges have been significantly flexed and will attempt to straighten back out. As the flanges attempt to reorient themselves linearly, they press against the interior walls of the nasal passages, causing the side portions of a person's nose to expand outward to the side. This causes the overall circumference of the nasal passages to expand. The air flow around the nasal breathing apparatus is virtually unrestricted, and thereby increases the efficiency of air transfer.

This invention is also able to be created using two guide plates that press a section of simple tubing flat, when the tubing is placed between the guide plates. These plates may operate independently, or may have a hinge configuration, where the plates pivot around a central point. If a hinge configuration is used, a pivot pin may be adjacent to the tubing between the hinge plates, or the pivot pin may be at a position away from where the tubing is pressed.

Once the tubing has been suitably pressed and held in a fixed position between the two plates, the shape of the plates will define notches and expose and portion of the tubing along the length of the end portions of said tubing. Once the exposed tubing is removed, either through punching it out or cutting it away, the resulting tubing will comprise the nasal breathing apparatus that is ready for use.

Accordingly, it is an object of this invention to provide a simple apparatus that will increase the internal circumference of a person's nasal passage.

It is a further object of this invention to provide an apparatus that will not restrict the air flow around the invention when in use.

It is a further object of this invention to provide an apparatus that is capable of being held in position through a strap or cord.

It is a further object of this invention to provide a nasal breathing apparatus that is capable of positioning an aromatic capsule or plug in close proximity to the nasal passage opening to maximize the aromatic benefits, be it for pleasure or for medicinal purposes.

It is a further object of this invention to provide a simplistic manner of making said nasal breathing apparatus, and for providing a template for the appropriate removal of various sections and portions of tubing.

### BRIEF DESCRIPTION OF THE FIGURES

FIG. 1 is a cross sectional view of the nasal breathing apparatus, as it is shown when placed into the nasal cavities.

FIG. 2 is a side view of the tubing as it appears prior to having any notches or side wall portions removed.

FIG. 3 is a cross sectional view of the tubing shown in FIG. 2, in which notches and a portion of the side wall ends have been removed to define the tubular body of the nasal breathing apparatus.

FIG. 4 is a perspective view of the tube portion shown in FIG. 3.

FIG. 5 is a cross sectional view of the nasal breathing apparatus, in which a cord is shown positioned through the first notch, the central tube portion, and exiting the tube through a second notch.

FIG. 6 depicts a view of a person with the the nasal breathing apparatus positioned within their nasal passages and held in place by the cord.

FIG. 7 is a cross sectional view of the nasal breathing apparatus with an aromatic capsule or plug placed within the central tube portion.

FIG. 8 is a cross-sectional view of the nasal breathing apparatus, having an aromatic capsule placed within the central tube portion, with the flexed end portions of the nasal breathing apparatus having been placed within nasal passages.

FIG. 9 is a top view of a plate, with a flattened tubular portion beneath.

FIG. 10 is a perspective view of a set of pivoting hinge plates that serve as a template for the method of making the tubular body of the nasal breathing apparatus, in which the pivot pin is located adjacent to the tubing.

FIG. 11 is a side view of the configuration shown in FIG. 10.

FIG. 12 is a bottom view of the nasal breathing apparatus, as seen with the outer ends flexed at a 90 degree angle, in relation to the central tubing portion, indicating the space available for the transfer of air.

### DETAILED DESCRIPTION OF THE INVENTION

I. The Breathing Apparatus

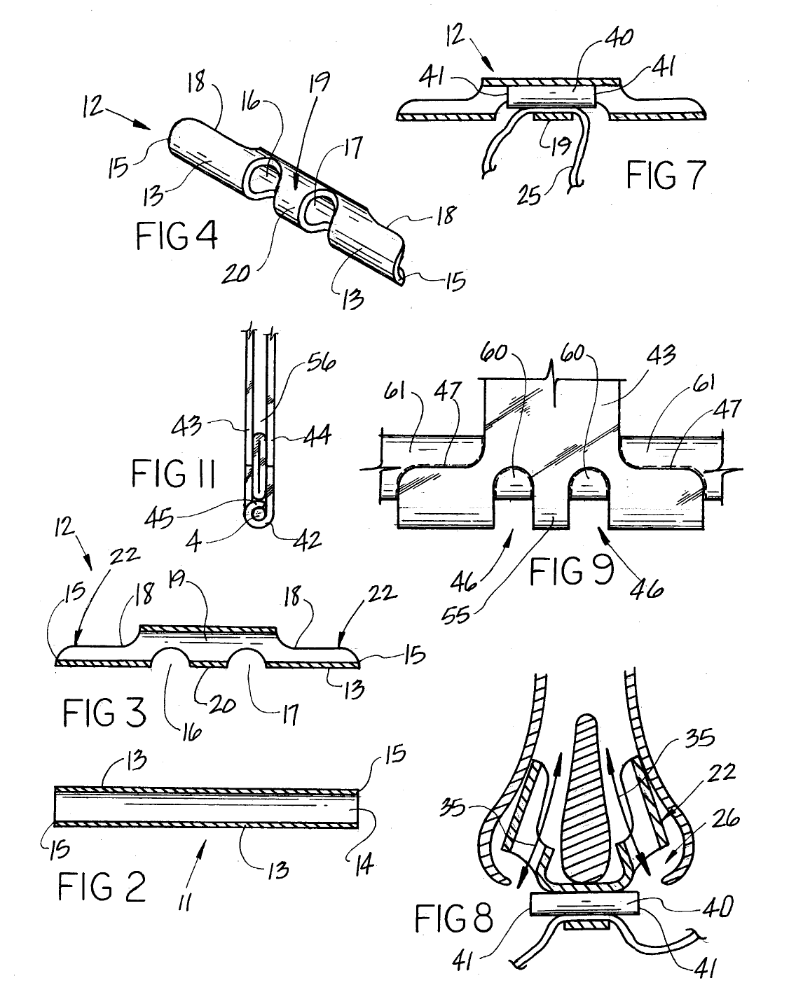

Referring now to FIG. 2, a length of ordinary plastic tubing 11 is shown. It should be understood that the tubing may comprise any material that is flexible, and capable of maintaining its consistency when exposed to moisture and the type of fluids typically found in human nasal passages. Said tubing 11 is comprised of a circular side tube wall 13 that encircles a central inner shaft area 14. The inner shaft area 14 is defined by the side tube wall 13 and the terminating ends 15 of said wall 13. As the nasal breathing apparatus 10 will need to be manipulated to a unshaped configuration, a means must be provided which will allow the length of the tubing 11 to be flexed or bent approximately 90 degrees or more, without causing the side wall 13 to deform or cause distortion. This type of deformity is typical in tubing that is bent or flexed 90 degrees or more, where the side walls tend to flatten and bulge outward.

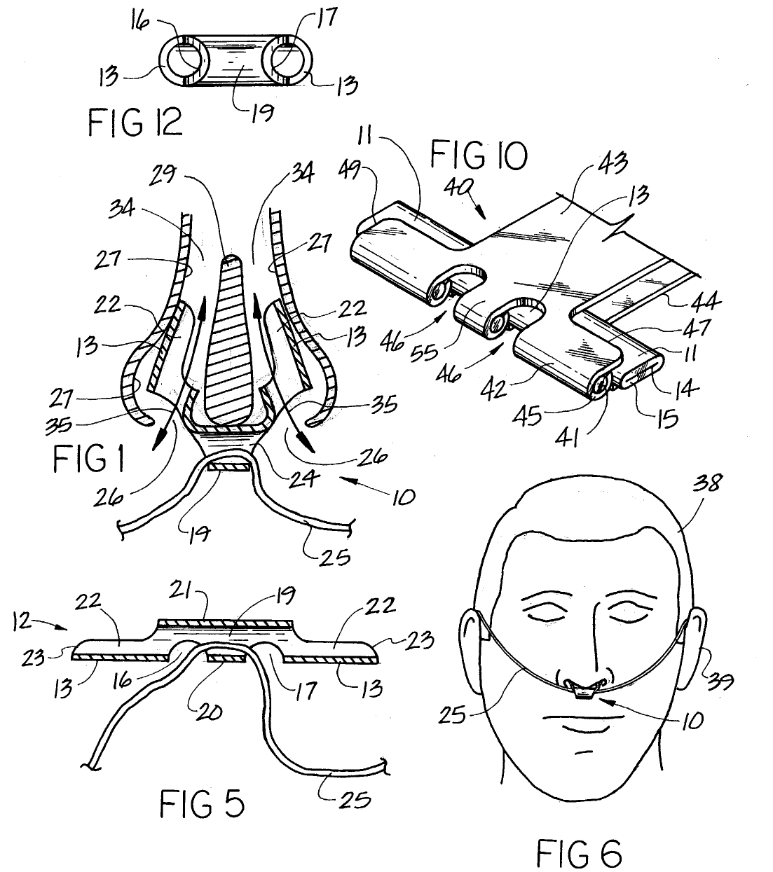

Referring now also to FIG. 3, the same tubing 11 as shown in FIG. 1 has been modified to the finished or modified tubing 12 as shown in FIG. 3. The finished tubing has had four separate areas of the side wall 13 removed. The first two areas described are the two notches 16 and 17, that comprise a basically somewhat circular opening through the side wall 13. Notches 16 and 17 are spaced apart by a central tube portion 19, with the side wall 13 between said notches 16 and 17 being defined as a central front side wall 20. Referring now also to FIG. 4, notches 16 and 17 are more clearly shown, as being separated by the central tube portion 19.

The second two areas removed comprise areas on the back side wall. The finished tubing 12, as shown in FIG. 3, also has a portion of side wall 13 removed on the back side of tubing 12. The back side wall of tubing 12 has been removed, at the point along the length of the tubing 12 from the outer edge of the respective notch 17 to the end 15, so as to define a back side end notch 18.

Likewise, the opposing back side wall of tubing 12 has been removed from the back side of the tubing 12, at a point along its length from approximately the outer side of notch 16 to the end 15. The portion of the tubing comprising a back side end notch and front side wall 13 comprise outer end 22, which is that portion of the tubing 12 which is placed within a person's nasal passageway.

Referring now also to FIG. 4, the the finished tubing 12 is shown, in which the front side of the tubing wall 13 is shown, having an arcual shape as depicted by the end 15. As is shown in FIG. 4, the ends 15 comprise only a portion of the previous circular tubing shape, as a result of the back wall portion having been removed. Approximately one half of the total circumference of the tubing has been removed along the length of said tubing, as compared with both ends of the original tubing member 11, so as to create the finished tubing 12. The benefits achieved with this modification is that there is an arcual side that is able to be presented to the nasal passageways, shown and described below, but since a portion of the circumference has been removed, there is no back side wall to restrict the flow of air through the nasal passages, as shown and described below.

As is also shown in FIG. 4, notches 16 and 17 comprise openings into the tubing 12. Although the figures indicate that the notches 16 and 17 are of sufficient size and shape so as to comprise the removal of circular shaped portions of the tubing, with the diameter of said circular shapes comprising approximately one half of the total circumference of tubing 12, the shape of the notches 16 and 17 may vary. For example, the notches 16 and 17 could also be defined as slits, cuts, or have any other reasonable shape that will allow portion of the total circumference of the tubing to be removed. Notches 16 and 17 are preferably adjacent to each other, and are divided or separated by the central tube portion 19, which comprises the bridge or separator portion of the tubing 12 that is placed underneath a person's nose, so as to allow the outer ends 22 to be directly placed within a person's nasal passage.

Referring now also to FIG. 5, the finished tubing 12 is shown, as a cross sectional view. A strap or cord 25 is shown, in which said strap 25 enters into the interior of the tubing 12 through a first notch 16, travels through the central tube portion 19, and exits out of said tubing 12 through a second notch 17. This strap 25 is an optional feature, whose benefits are shown in FIG. 6, while the nasal breathing apparatus 10 is being worn.

The nasal breathing apparatus 10 comprises the finished tubing, which has been flexed or bent so that the outer portions 22 are oriented at least 90 degrees as compared to the central tubing portion 19. When both outer portions 22 are so flexed or bent, the apparatus 10 forms a generally u-shaped or v-shaped configuration. Use of the apparatus is accomplished by placing the outer tubing portions 22 through nasal openings 26 and into its respective nasal passageway 34. In this manner, one outer tubing portion 22 will be placed in each of the nasal passageways 34. The apparatus 10 will attempt to straighten out, and the outer tubing portions 22 will press outward. The front side of the tubing wall 13, which has an arcual surface, will impact against the nasal interior side wall 27, and urge it away from the nasal septum 29. This will cause an increase in the volume of the nasal passageway 34. Since the front arcual side wall 13 provides a broad surface, there is minimal discomfort. Air flow 35 through the nasal openings 26 and deeper into the nasal passageways 34 is unrestricted by this apparatus 10. As FIG. 12 indicates, the air flow through the tubing is virtually unrestricted, when the tubing 12 has been bent into a u-shaped configuration. The air flow 26 is able to move through the exposed tubing, as a result of the notches 16 and 17 defined along the finished tubing 12. The notches 16 and 17 allow the tubing 12 to bend to the necessary angles, without any undue deformation of the tubing 12 as would typically be seen without any notches 16 and 17 being present.

This apparatus 10 has the additional benefit of being able to maintain its position within the nasal passageways 34 by virtue of the large surface area available on the tubing front side 13, and by virtue of the fact that the consistent pressure along the length of the outer tubing portions 22 is available along their length inside the nasal passageway 34 against the interior nasal wall 27. As FIG. 6 shows, the strap 25, as shown in FIG. 5 and FIG. 1 is able to encircle a person's head 38, or even hook around the ears 39. The cord or strap 25 is in a sense attached to the finished tubing 12, but only in the sense that it is not removable unless slid out of the tubing 12. It should be understood that this cord or strap 25 may be elastic, or may be resistant as to stretching. The strap 25 may be physically attached to the tubing 12, such as being stapled, snapped, hooked, glued, or any other permanent means.

Referring now also to FIGS. 7 and 8. The finished tubing 12 of the nasal breathing apparatus provides a means in which aromatic substances may be placed adjacent to nasal openings. FIG. 7 depicts an aromatic capsule 40 that has been placed within the central tubing portion 19. As the central tubing portion 19 maintains a constant circumference and volume, even when the tubing 12 forms a u-shape, an aromatic capsule 40 is able to be inserted and held in place during manipulation of the outer end portions 22. Further, as FIG. 8 shows, the aromatic capsule 40 continues to maintain position in the central tubing area 19, with the ends 41 of the aromatic capsule 40 protruding slightly into the air flow 35. The aromatic capsule 40 may comprise any substance or media commonly used for the slow release of aromatic compounds. Substances such as formed plastic foam, spongy materials, and porous materials that are capable of receiving aromatic compounds, may be used. The aromatic capsule 40 is depicted in FIG. 7 and FIG. 8 as a cylindrical shape whose outer circumference is similar to the inner circumference of the central tubing area 19. FIG. 8 depicts how the outer tubing portions 22 have been bent upward, allowing unrestricted access to the central tubing area 19, in which the aromatic capsule 40 is then able to be simply slid in to the central tubing area 19. Removal of said aromatic capsule 40 is easily accomplished by either pulling said capsule 40 out of the central tubing area 19, or pushing it through the central tubing area 19 until it can be retrieved or dropped out of the other end of the central tubing area 19.

This aromatic capsule 40 may comprise other shapes and configurations, and may be held in place through friction between the aromatic capsule's outer surface and the central tubing area 19, or may be maintained in the proper position through any other means commonly known and understood in the art. The aromatic capsule 40 may be permanently fixed to the central tubing area, or have a shape or configuration that promotes stability within the central tubing area in 19. The benefits of the aromatic capsule 40 comprise the ability to introduce chemical aids or substances to the breathing passageways. Chemical substances that promotes breathing are readily able to be introduced, as well as odors that are simply pleasing to the wearer. Medication of certain types, may even be administered in this matter. Since there is virtually no appreciable distance between the aromatic capsule and the nasal opening 26, there is no appreciable loss of airborne substances that would otherwise be lost through atmospheric dispersal. This invention provides the most efficient means to externally deliver aromatic substances to the nasal openings 26.

II. The Method of Modifying Tubing Into the Apparatus

Referring now FIGS. 9-11, a method of modifying tubing 11, as shown in FIG. 2, is shown. In order to modify the tubing 11 into the tubing 12 that has had areas or portions of the side wall 13 removed, a novel method is shown. The first step of this method is to acquire a length of plastic tubing 11, such as shown in FIG. 2 and place it between two opposing plates 43 and 44, as shown in FIG. 9-11. The opposing plates 43 and 44 have a defined shape, and they serve as a template for removing exposed tubing 11 that is placed between said plates 43 and 44. In order to accurately remove the necessary portions of the tubing 11, the second step is to allow said tubing 11 to be pressed between plates 43 and 44, as they are urged together, which cause said tubing 11 to be flattened as it is squeezed between said plates 43 and 44.

Plates 43 and 44 may be independent of each other, moving toward each other in a linear fashion, such as would be found in a press. Plates 43 and 44 may also pivot around a hinge means, with said plates 43 and 44 pivoting toward each other as they squeeze the tubing 11. FIG. 9 is intended to show that top view of plate 43, with a hidden plate 44 directly beneath plate 43, so that plate 44, which is identical in shape to plate 43, is completely obscured from view by plate 43. Plate 43 and plate 44 have an identical shape, regarding that portion of said plates 43 and 44 that engage or contact tubing 11.

Both plate 43 and plate 44 define notch areas 46, which are defined and spaced apart by a central flange 55. As it is shown in both FIG. 10 and FIG. 9, the exposed portion of the tubing 11 that comprises that portion of the tubing wall 13 that will be removed to create notch 16 and notch 17 is exposed. Like wise, when said tubing 11 is pressed between plates 43 and 44, the portion of the back wall 13 that will be removed to create and define the peripheral notches 18 is shown and exposed. For clarity, FIG. 9 indicates the areas of the tubing intended for removal, as areas 60 and 61. Removal of the exposed portion of the tubing 11 may be accomplished through use of a punch that engages the plastic tubing in a manner so that the exposed tubing is sheared off during the movement of the punch apparatus past plates 43 and 44. The punch apparatus would comprise a matching member that corresponds in a manner that is opposite to the shape of plates 43 and 44. For example, a punch means having a rounded portion that matches the shape of the notch 46, defined by a plates 43 and 44, would move through said notch 46 and punch out and remove the exposed portion of the tubing 60, as shown in FIG. 9. It is a similar action with a mating shape punch part would remove the exposed portion of tubing 61, shown in FIG. 9. Once portions 60 and 61 are removed, plates 43 and 44 move away from each other, allowing the tubing to expand return to its original circular shape, with the resulting tubing now comprising the modified tubing 12 as shown in the other respective figures.

In addition to the use of the punch, any type of cutting means may be used to shear off and remove tubular portions 60 and 61. Cutting means such as blades, curved sharpened edges, and other types of cutting means may be used, that follow the configuration offered by plates 43 and 44.

Examples of the involvement of plate 43 and 44, as noted above, may also comprise a hinge means. As is shown in FIGS. 10 and 11, the hinge means, comprising a hinge edge 42, that encircles a hinge pin 41 is shown. Said hinge edge 42 effectively operates as a tube stop 45, serving as a guide against which the length of tubing 11 may be pressed against, so as to properly orient tubing 11 between plates 43 and 44. In this configuration, plates 43 and 44 rotate and pivot around the pivot pin 41, and compress tubing 11 as they move toward each other. The hinge edge 42 may operate as the control for optimal thickness or space 56 allowed between plates 43 and 44, so that when plates 43 and 44 are parallel to each other, as shown in FIG. 11, said plates 43 and 44 will have squeezed the tubing 11 to the optimal flattened shape.

It should also be understood that hinge means may also be located away from said tubing 11, with the plates 43 and 44 pivoting around a hinge pin 41 that is a distance away from where the tubing 11 is situated between plates 43 and 44. In this configuration, it is necessary for the tubing 11 to be placed between plates 43 and 44 in a manner by which its placement can be controlled and properly directed, so that the plates 43 and 44 engage said tubing 11 in the proper position so as to allow the exposure of tubing portions 60 and 61.

From the foregoing statements, summary and description in accordance with the present invention, it is understood that the same are not limited thereto, but are susceptible to various changes and modifications as known to those skilled in the art and we therefore do not wish to be limited to the details shown and described herein, but intend to cover all such changes and modifications which would be encompassed by the scope of the appended claims.

I claim:

1. A breathing apparatus that improves the ease of air flow through a person's nasal passageways, caused by the physical enlargement of the inner circumference of a person's nasal passage, comprising:
a. a length of resilient tubing, having a tube body defined by a central tubular portion positioned between two separate ends, where said tubing is capable of being bent into a u-shape, and where said ends are able to be positioned within a wearer's respective nasal passageways;
b. a structural modification to the tubing that allows bending of the tubing without distortion to the tube body; and
c. a means to allow unrestricted air flow through the tube when the distal end is bent at an angle in relation to the central tube portion.

2. A breathing apparatus that improves the ease of air flow through a person's nasal passageways, as recited in claim 1, in which the structural modification to the tubing that allows bending comprises notches, that are defined on the front side of the tubing and which are separated by the central tubular portion.

3. A breathing apparatus that improves the ease of air flow through a person's nasal passageways, as recited in claim 1, includes a strap that is attached to the tubular portion of the apparatus, and which is capable of encircling the user's head.

4. A breathing apparatus that improves the ease of air flow through a person's nasal passageways, as recited in claim 1, in which a strap is threaded through the central tubing area, with said strap entering through a first notch, extends through the central tubular area, and exits out of a second notch, and where said strap is capable of encircling the user's head.

5. A breathing apparatus that improves the ease of air flow through a person's nasal passageways, as recited in claim 1, in which the back side of the tubing defines peripheral notches on both ends of the tubing, and where the front side of said tubing body is able to press against the nasal passageways when inserted into a wearer's nasal passageway, and where said front side presents an arcual surface.

6. A breathing apparatus that improves the ease of air flow through a person's nasal passageways, as recited in claim 1, in which said apparatus includes a means to hold said apparatus in place, while being worn by a person.

7. A breathing apparatus that improves the ease of air flow through a person's nasal passageways, as recited in claim 1, in which said apparatus include an aromatic capsule that is placed within the central tubular portion.

8. A breathing apparatus that improves the ease of air flow through a person's nasal passageways, through the physical enlargement of the inner circumference of a person's nasal passage comprising:
a. a length of resilient tubing, having a tube body defined by a central tube portion positioned between two separate ends, where said tubing is capable of being bent into a u-shape, and where said ends are able to be positioned within a wearer's respective nasal passageways;
b. notches on the front side of the tubing, comprising circular openings defined along the length of the tubing at the point where the tube body is intended to be bent, and which provide openings into the nasal passageways for air;
c. means to hold the apparatus in place within the nasal passageway; and
d. means for receiving and holding an aromatic capsule.

9. A breathing apparatus that improves the ease of air flow through a person's nasal passageways, as recited in claim 8, in which a strap is threaded through the central tubing area, with said strap entering through a first notch, extends through the central tubular area, and exits out of a second notch, and where said strap is capable of encircling the user's head.

10. A breathing apparatus that improves the ease of air flow through a person's nasal passageways, as recited in claim 8, in which the means to hold the apparatus in place comprises an elastic strap that is attached to the tubular portion of the apparatus, and which is capable of encircling the user's head.

11. A means to modify plastic tubing into a usable breathing assistance apparatus for use in nasal passageways, comprising the following steps:
a. placing a length of plastic tubing between two opposing plates that have defined edges or notches that engage the front side of the plastic tubing, and which have an edge that allows a portion of back side of the plastic tubing to be exposed;
b. pressing the plates together until the tubing is flattened between said plates, with a portion of the tubing being exposed;
c. removing the exposed tubing that extends outward from the side of the plates; and
d. separating the plates to allow the tubing to return to its original tubular configuration.

12. A method to convert plastic tubing into a breathing apparatus for use in nasal passageways, as recited in claim 11, in which the opposing plates move independently of each other, and engage each other at a predetermined position.

13. A method to convert plastic tubing into a breathing apparatus for use in nasal passageways, as recited in claim 11, in which the opposing plates pivot around a hinge pin assembly that is adjacent to the tubing placed between the plates, and where the hinge pin assembly provides a stop for the placement of said tubing against the hinge pin assembly.

14. A method to convert plastic tubing into a breathing apparatus for use in nasal passageways, as recited in claim 11, in which the opposing plates pivot around a hinge pin assembly that is adjacent to the tubing placed between the plates, and where the hinge pin assembly is on the end of the plates opposite of the end that the tubing is being placed.

15. A method to convert plastic tubing into a breathing apparatus for use in nasal passageways, as recited in claim 11, in which the exposed tubing is removed by using a punch apparatus that moves perpendicular to the plates, and engages the plastic tubing in a manner so that the exposed tubing is sheared off during the movement of the punch apparatus.

16. A method to convert plastic tubing into a breathing apparatus for use in nasal passageways, as recited in claim 11, in which the exposed tubing is removed by using a cutting means to shave the exposed plastic away from the tubing held between the plates.
